(19) United States
(12) Patent Application Publication
(10) Pub. No.: US 2002/0007504 A1
(43) Pub. Date: Jan. 17, 2002

(54) Hybrid tea rose plant named 'Keihatakaho'

(76) Inventor: Takashige Ishikawa, Aichi-Ken (JP)

Correspondence Address: BURNS DOANE SWECKER & MATHIS L L P, POST OFFICE BOX 1404, ALEXANDRIA, VA, 22313-1404, US

(21) Appl. No.: 09/832,840

(22) Filed: Apr. 12, 2001

(30) Foreign Application Priority Data
Jul. 6, 2000 (JP) 12635

(51) Int. Cl. A01H 5/00

(52) U.S. Cl. PLT/137

(57) ABSTRACT

A new and distinct variety of Hybrid Tea rose plant is provided which abundantly forms attractive deep pink blossoms. The blossoms commonly possess approximately 50 petals each. The buds are large and are borne on straight erect stems. Attractive dark green semi-glossy foliage is formed. The new variety is particularly well suited for forming cut flowers under greenhouse growing conditions.

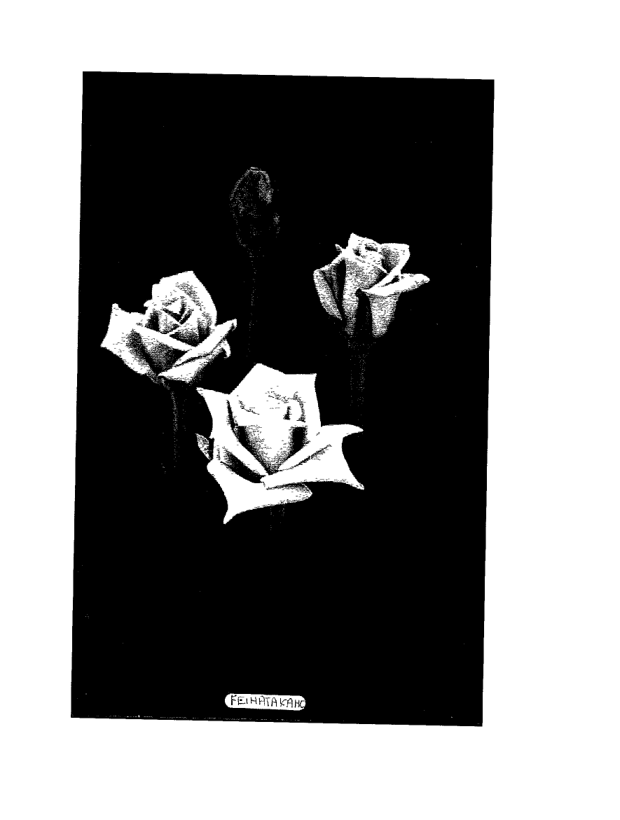

### SUMMARY OF THE INVENTION

The new variety of Rosa hybrida Hybrid Tea rose plant of the present invention was discovered during 1998 in Japan while growing among plants of the ‘Febesa’ variety (U.S. Plant Pat. No. 11,529). The new variety of the present invention accordingly is believed to be a naturally-occurring mutation of the ‘Febesa’ variety of unknown causation.

The new variety was selected and preserved primarily because of its distinctive blossom coloration that differed from the parent ‘Febesa’ variety. More specifically, the ‘Febesa’ variety exhibits blossoms that are light pink in coloration whereas the new variety of the present invention forms blossoms that are a deeper pink in coloration. In addition, the number of petals per blossom is different. More specifically, the ‘Febesa’ variety commonly forms approximately 20 to 24 petals per blossom on average whereas the new variety of the present invention commonly exhibits approximately 50 petals per blossom on average.

It was found that the new variety of Hybrid Tea rose plant of the present invention possesses the following combination of characteristics:

(a) forms attractive large buds on straight erect stems,

(b) forms in abundance deep pink blossoms,

(c) forms attractive dark green semi-glossy foliage, and

(d) is particularly well suited for the production of cut flowers under greenhouse growing conditions.

The new variety well meets the needs of the horticultural industry and can be used to advantage during the commercial production of cut flowers under greenhouse growing conditions.

The new variety of the present invention also can be distinguished from the ‘Keihayanasa’ variety (U.S. Plant Patent Application Ser. No. ______, filed concurrently herewith) that also is a mutation of the ‘Febesa’ variety. More specifically, the ‘Keihayanasa’ variety forms salmon pink blossoms having approximately 30 to 34 petals per blossom.

The new variety of the present invention has been found to undergo asexual propagation by a number of routes, including budding, grafting, and the rooting of cuttings. Such asexual propagation by the above-mentioned methods as performed in France has shown that the characteristics of the new variety are strictly transmissible by such asexual propagation from one generation to another.

The new variety has been named the ‘Keihatakaho’ variety.

### BRIEF DESCRIPTION OF THE PHOTOGRAPH

The accompanying photograph shows as nearly true as it is reasonably possible to make the same, in a color illustration of this character, typical specimens of the flower of the new variety wherein the bud is shown together with flowers in various stages of opening. The depicted plant was being grown in a greenhouse at Chiba-Ken, Japan during May.

### DETAILED DESCRIPTION

The chart used in the identification of the colors is the R.H.S. Colour Chart of The Royal Horticultural Society, London, England. The terminology which precedes reference to the chart has been added to indicate the corresponding color in more common terms. The description is based on the observation of two year-old specimens of the new variety during April while budded on Rosa indica understock and growing in greenhouses at Le Cannet des Maures, Var, France.

Class: Hybrid Tea.

Plant:

Height.—when pruned to height of 0.85 cm, floral stems having a length of approximately 50 to 80 cm commonly are produced at the end of the growing season.

Habit.—erect.

Branches:

Color.—young stems: Yellow Green Group 146A. —adult wood: Green Group 137A.

Thorns.—size: medium. —quantity: numerous. —color: Greyed-Orange Group 167A.

Leaves:

Stipules.—adnate, pectinate and narrow.

Petioles.—near Yellow-Green Group 147A to 147D, and somewhat glandular.

Leaflets.—number: 3, 5 and 7 (most often). —shape: elliptic. —serration: regular. —texture: somewhat consistent. —general appearance: very dense, dark green and semi-glossy foliage.

color (young foliage): upper surface: near Yellow-Green Group 147A. under surface: near Yellow-Green Group 147B. —color (adult foliage): upper surface: near Green Group 139A. under surface: near Green Group 138A.

Inflorescence:

Number of flowers.—commonly one per stem.

Peduncle.—near Green Group 138B and the length is approximately 8.5 cm on average.

Sepals.—near Green Group 137D in coloration and with some extensions as illustrated in the photograph.

Buds.—shape: elongated. —size: large. —length: approximately 5.5 cm on average.

Flower.—shape: commonly with a high center. —diameter: approximately 9 cm on average. —color (when opening begins): upper surface: deep pink, near Red-Purple Group 65A to 65C. under surface: deep pink, near Red-Purple Group 65B to 65C. —color (when blooming): upper surface: deep pink, near Red-Purple Group 65A to 65C. under surface: deep pink, near Red-Purple Group 65B to 65C. —color (at end of opening): upper surface: deep pink, near Red-Purple Group 65A to 65C. under surface: deep pink, near Red-Purple Group 65B to 65C. —fragrance: none. —lasting quality: excellent when cut and placed in a vase with the blossoms commonly lasting approximately 15 days. —petal shape: rounded with an obtuse base and relexed edges. —petal number: approximately 50 on average. —stamen number: approximately 106 on average. —anthers: ochre in coloration. —filaments: yellowish in coloration. —pistil number: approximately 146 on average. —stigmas: yellow-strawlike in coloration. —receptacle: light green in coloration, smooth, and in longitudinal section in the shape of funnel.

Development:

Vegetation.—very vigorous.

Blooming.—very abundant.

Resistance to diseases.—excellent especially with respect to Powdery Mildew, and Black Spot.

I claim:

1. A new and distinct variety of Hybrid Tea rose plant characterized by the following combination of characteristics:
(a) forms attractive large buds on straight erect stems,
(b) forms in abundance attractive deep pink blossoms,
(c) forms attractive dark green semi-glossy foliage, and
(d) is particularly well suited for the production of cut flowers under greenhouse growing conditions;
substantially as herein shown and described.
